(19) United States
(12) Patent Application Publication
(10) Pub. No.: US 2010/0076528 A1
(43) Pub. Date: Mar. 25, 2010

(54) DystoFit / NeckFit

(76) Inventor: Andrea Maria Zeller, Bolzano (IT)

Correspondence Address: Christian Zeller, 1605 Gulf Road, Tarpon Springs, FL, 34689, US

(21) Appl. No.: 12/610,398

(22) Filed: Nov. 2, 2009

(30) Foreign Application Priority Data
Mar. 19, 2007 (DE) 20 2007 003 980.8
Apr. 13, 2007 (AT) GM 237/07

(51) Int. Cl. A61N 5/067; F21V 21/08; A61N 5/06

(52) U.S. Cl. 607/89; 362/103; 607/90

(57) ABSTRACT

A battery powered, vertically adjustable point/line, or cross laser module device, affixed leveled to a clip on the center of an eyeglasses bridge for the treatment of cervical dystonia; squint angle diagnostic; ADHD eye-hand coordination; golf player head posture correction; cervicogenic headache tension relief; neck pain treatment & neuromuscular and neuro-physiological re-education. By directing the cross laser beams at predetermined reference areas, patients are offered a visual feedback and respectively a visual response in an effective body posture and/or a motion sequence with the purpose of learning a coordinated motion sequence.

This invention refers to a device for the treatment of cervical dystonia and the other above mentioned; with the aid of this device, the patients are offered a visual feedback and respectively a visual response in an effective body posture and/or a motion sequence with the purpose of learning a coordinated motion sequence.

Cervical dystonia, that is a dystonia that involves the throat/neck area, also known as torticollis spasmodicus, distinguishes itself through an involuntary abnormal head posture and head motion, induced by over-active throat and neck muscles. During this process, the normal coordination between individual muscles and their opponents is so severely disturbed that these muscle groups are mistakenly activated at the same time. In most cases, the head cannot be actively brought from this deviation into the normal posture. There are various basic types of cervical dystonia, which distinguish themselves through a different type of movement rhythm and a different direction of the head deviation. Thus, we can distinguish several types: the spinning, rotating Torticollis, the Laterocollis tilting to the shoulder, the Anterocollis bending forward toward the chest, and the Retrocollis overstretched backwards. Moreover, shifts of the head-body axis occur on several levels, so that the patients involved experience at times a relocation of their body axis of approximately 15° toward the center.

There are also symptomatic dystonia types, whose cause is known if for instance certain brain centers have been damaged by disorders. However, no cause has been determined for almost all types of focal dystonia, and thus also for torticollis, and in this case, this is called an idiopathic dystonia.

The cervical dystonia is caused presumably in the brain by a disturbance of the automatic control of the head posture.

With the exception of the symptomatic cervical dystonia types, which can be treated primarily based on their cause, there are no curative methods to date for cervical dystonia. The most effective treatment method so far is the treatment based on botulinum toxin. A certain group of muscles is temporarily partially paralyzed in this process. The effect lasts around 3 months.

Dystonia patients often seek alternative treatment methods because the response to botulinum toxin is not sufficient or they refuse it. So far, the alternative treatments include acupuncture, relaxation methods, and homeopathic treatments such as detoxification. None of these alternative treatments have been able to produce so far a lasting relief of the symptoms associated with dystonia.

With the so-called writer's cramp and respectively musician's cramp, which likewise belongs to the category of focal dystonia, it has been found that the finger control areas are overlapped in the brain (Altenmüller E. et al., Hand Clin 19 (2003), 1-6). The fingers that experience cramps in different situations of conscious movement do not have any more a representation area in the cortex; these areas are overlapped by the representation areas of the fingers that move normally. As soon as these fingers are moved, the fingers that do not have a representation area in the cortex anymore are moved at the same time involuntarily dystonically (crampingly).

The SMR method (=sensory motor returning; Candia V. et al., The Lancet, Band 353, page 42) can produce some relief in this case, which essentially has to do with a movement therapy concept for treating the symptoms. Working fingers remain still, and sensomotor exercises are performed with the otherwise spastic fingers. It has been determined that the control areas in the brain tend to separate.

For the development of the device described in this invention, it has been researched which movement components are disturbed and which work normally on an eye and head movement and respectively when grabbing an object. Since the control areas of the eyes, neck, and hands lie side by side in the motor cortex very close to each other, it has been assumed that an overlapping of the control areas may occur here as well. As shown in C. Maurer et al. (Brain (2001), 124, 413-426), the eye movement is not disturbed in the case of cervical dystonia.

Now, this invention is based on the knowledge that the patient should be able to keep his/her eyes still for a possible therapy for cervical dystonia. The device used for the therapy of cervical dystonia was developed based on this knowledge, which includes primarily a light source designed to generate an optical sign visible in daylight and a power source to supply the light source with power; additionally, the device features design characteristics which basically place the light source between the eyes of the person who is undergoing therapy.

With this device, it becomes possible to fixate the eyes constantly while a light source is applied to the head, respectively between the eyes; the eyes fixate on the light beam respectively light point during each head movement.

With such a device, the test person, who had not been able for more than a year to direct her look to the right because her head deviated to the left, became immediately able to turn her look and head to the right effortlessly. According to a yet unpublished study which involved ten test persons and was performed at the Neurological University Hospital in Innsbruck, it was possible to achieve, after a six-week practice phase, an improvement of the symptoms of approximately 50%.

The major characteristics of this device that are described here, according to this invention, emerge as a result of the following requirements:
The light source must be at the level of the eyes.
A perfectly punctiform light is needed to practice the eyes-head movement coordination and respectively eyes-head-hand movement coordination.
A linear light source is necessary to practice the body posture, respectively the centering of the head (perpendicularly).
The light must be visible in daylight since the device must be used for everyday movements and/or operations, and respectively activities.
The light source, respectively the light beam and respectively the light point should be pivotable upwards or downwards, if necessary, because the light beam must be adjusted according to the normal eye position/movement. Thus, for example, when reading, the eyes are lowered downward at a certain angle, and so we should be able to tilt accordingly the light beam, respectively the light source.

Consequently, to adjust the light source, a retainer must be provided to receive and adjust the light source, which is a sidepiece, placed preferably on the nose and/or ears of the person who is undergoing therapy. If needed, the retainer, respectively the support piece may also be a pair of glasses or may come in the form of a pair of glasses.

The light source emits preferably colored light, which is easy to see in daylight. The particularly preferred light source is a laser light source, which preferably emits a strictly punctiform light for practicing the coordination of eyes-head movements, respectively eyes-head-hand movements. For this purpose, a point laser module TIM-230-3 may be used, with a tight wavelength range of 650 up to 660 nm and a power consumption of <40 mA. To practice the posture and respectively the centering of the head, a linear light source is applied, especially a laser light source with a linear beam laser module, for example the linear beam laser module TIM-230-9 with a wavelength of 635 nm and a power consumption of <50 mA.

For various application options, it is required that the light source be connected with the retainer and respectively the mounting element in a pivotable manner. It is more advantageous to have the light source fully adjustable upwards and downwards, so that it can be adjusted according to the direction of the look (or coaxially to the direction of the look).

In an additional preferred embodiment, the light source is adjusted as pivotable on a clip-mount, so that the light source may be placed on each regular pair of glasses with a solid rim.

The power source is preferably one that can be connected with the light source or a connected battery. Preferably, this power source may be integrated in the form of a mini-battery, for instance also in the retainer or in the clip-mount.

This device, that makes the object of this invention, is further explained in detail below, using drawings, which show preferred embodiments of this invention:

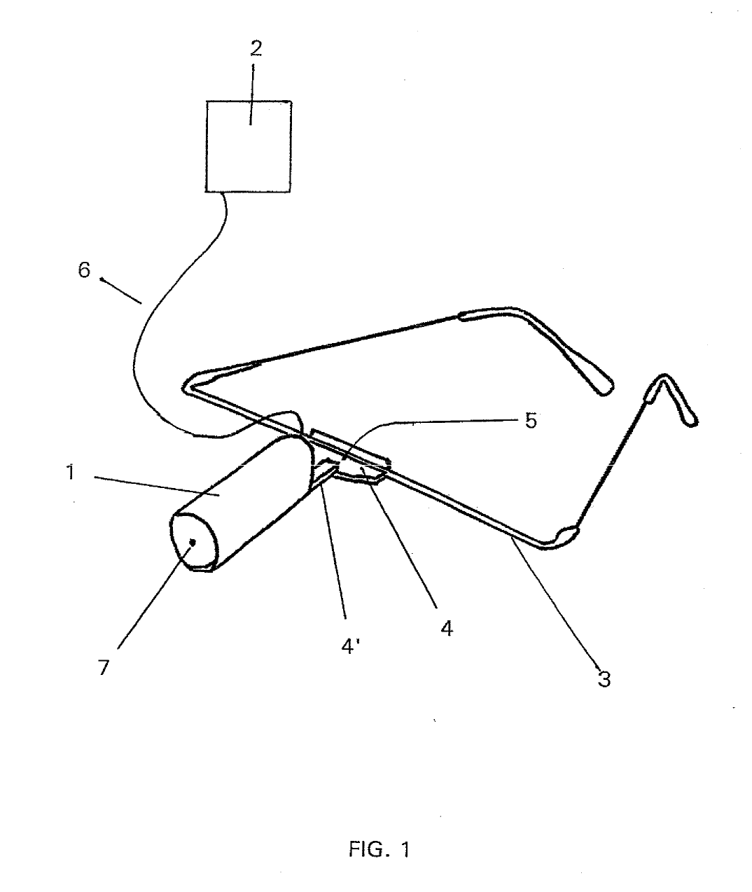

FIG. 1 is a schematic representation of a device, based on this invention, for the therapy of cervical dystonia. FIG. 1 represents a three dimensional view of the device from the frontal right.

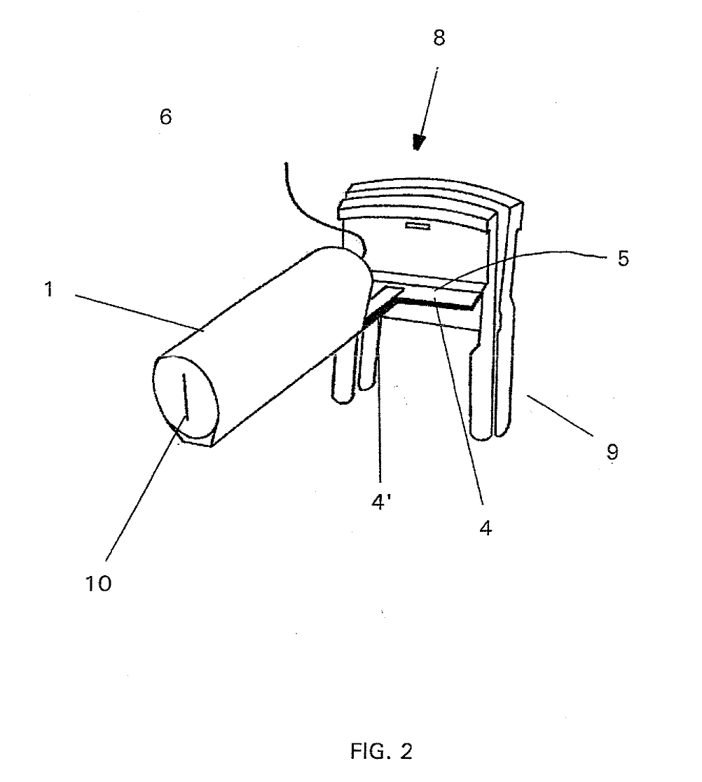

FIG. 2 is a mounting variant of an additional preferred embodiment. FIG. 2 represents a three dimensional view of the device from the frontal right.

FIG. 1 represents all the essential elements of the device, according to this invention, which can be employed for the therapy of cervical dystonia. Based on the simple design elements, which are in general commercially available, the construction of the device is simple and therefore cost-effective, which is not an insignificant factor for therapy purposes.

FIG. 1 shows therefore a light source 1, which is connected to a retainer 3. The retainer 3 is represented here as a sidepiece of a pair of glasses, and the light source 1 is connected to the retainer 3 in the area of the nose side piece (which must not be seen). Therefore, the light source 1, when used, is always at eye height.

The connection between the light source 1 and the retainer 3 is achieved in this case through a connector 4, which on the one hand can be held un-detachable to the retainer 3 and on the other hand features a receiving element 4′, from which the light source 1 can be received in an undetachable manner. The connector 4 may be fully connected with the retainer 3 or can thereby be brought into an operative interaction through a type of break-clip mechanism. The connector 4 features a tilt element 5, or such a part is located between the connector 4 and the receiving element 4′, with which the light source 1 is pivotable vertically on the retainer 3 (see the double arrow) upwards or downwards. Thereby, the light beam emitted by the light source 1 is adjusted according to the normal eyes position/movement, which for example while reading is different from looking straightforward.

In the embodiment shown on FIG. 1, the light source 1 is a laser light source, which furthermore is equipped with a point laser module 7. This may involve an ELV¹-generated laser light source with point laser module TIM-230-3. This features a length of 18 mm and a diameter of 9.1 mm. These dimensions are not restrictive, but they prevent a dysfunction in the patient's natural field of vision. The laser source works in a tight wavelength range of 650 up to 660 nm and shows a power consumption of <40 mA. Of course, a linear beam laser module is also usable here if the therapy type requires it. The light source 1 is connected respectively connectable to an independent battery through cable 6. The preferably 3 to 4.5 V-battery is lowered into a specifically adjusted box, which is worn by the patient while exercising on a strap around the neck or by a clip attached to the waist belt. ¹ELV stands for Elektronik Literatur Verlag, a mail-order company for electronic devices.

For this reason, the device shown on FIG. 1 for the therapy of cervical dystonia meets all the above-mentioned requirements, since the light source is placed at eyes height, the light is visible in daylight, and a strictly punctiform light is provided for exercising the coordination of eyes-head movements, respectively eyes-head-hand movements; furthermore, the light source, respectively the light beam generated by it, is pivotable upwards and/or downwards.

A linear light source 1′, which is desirable for certain applications, is shown on FIG. 2 in relation to a particular mounting version of this invention.

Particularly, FIG. 2 shows a commercially available clip-mount 8 with clip legs 9. Thereby, the clip-mount 8 may be placed on each regular tight glasses frame. This embodiment is particularly suited to patients who need a vision aid. The connector 4 with the tilting element 5 stands out vertically to the clip-mount 8. A light source 1 is placed on the connector 4 over the receiving element 4′, preferably a laser light source again, and with a linear beam laser module 10 as represented here. The linear beam laser module, which again can be ELV-generated (TIM-230-9), features a length of 30 mm and a diameter of 10.5 mm and works exactly with a wave length of 635 nm. It has a power consumption of less than 50 mA. Of course, a point beam laser module can also be used here instead of a linear beam laser module.

The clip-mount 8 may be produced, for example, by the Eschenbach Company (Labo med 1646²).

The light source l' may be, as previously, connected or connectable to a battery or similar power-supplying element.

### APPLICATION

With the device that makes the object of this invention, a coordination training of the movement of the eyes, of the head/neck, and, if needed, of the hand is specifically carried out. By fixating the eyes on the light point visible in daylight or likewise on the light indicator, the patient involved in this process acquires first-hand information about his/her head/neck position, which enables a lasting correction of the faulty posture through a gradual learning of the eyes and head coordination and respectively of the eyes, head, and hand coordination.

Therefore, the device may be used in general everyday situations, for example when writing a letter, while eating or handling other equipment, which eases quite considerably the everyday life of cervical dystonia patients. Moreover, a sustained improvement of the symptoms occurs as a result of the already addressed learning effect.

A significant advantage of this device, based on this invention, is that the treatment that takes place therewith and respectively the training carried out therewith have absolutely no side effects, as opposed to the application of botulinum toxin.

Other application areas can also be conceived for this device, based on this invention. For example, the linear beam laser may be connected anywhere on the body, and this provides a visual feedback (back coupling) about the actual body posture and enables this way a correction of unconscious faulty postures.

Certain disorders, respectively injuries of the brain, may potentially lead to body schema disturbances. The subjective center is perceived as shifted, respectively displaced, which considerably impairs the general motion sequence as well as the balance. The visual feedback to a linear beam laser, which in this case should be also placed at eyes height, enables the patient to re-learn a sense of the center and inherently his/her balance.

The re-learning of the eyes, head, and body movement coordination is of vital importance for various types of dizziness and vertigo.

Both a device equipped with a point laser as well as one that features a linear beam laser, based on this invention, may be successfully used for balance training therapy.

In addition to the treatment of cervical dystonia, the device specifically lends itself to the treatment of squint angle diagnostic (point laser module); ADHD eye-hand coordination (point laser module); and golf player head posture correction (line laser module).

1. Device for cervical dystonia therapy, squint angle diagnostic, ADHD eye-hand coordination, golf player head posture correction, which includes primarily,
a light source (1) designed to generate an optical sign visible in daylight and
a power source (2) to supply the light source (1) with power
whereby the device features additional design characteristics, which involve placing the light source (1) mainly between the eyes of a patient who is undergoing therapy. The device, based on claim 1, is characterized by the fact that in order to adjust the light source (1), a retainer (3) is provided to receive and adjust the light source (1). The device, based on claim 1 or 2, is characterized by the fact that the retainer (3) is a sidepiece placed on the nose and/or ears of the person undergoing therapy. The device, based on claim 3, is characterized by the fact that the retainer (3) is a pair of glasses. The device, based on claims 1 through 4, is characterized by the fact that the source light (1) produces colored light. The device, based on claims 1 through 5, is characterized by the fact that the light source (1) is a laser light source. The device, based on claim 6, is characterized by the fact that laser light source (1) includes a point laser module. The device, based on claim 6, is characterized by the fact that the laser light source includes a linear beam laser module. The device, based on one of the previous Claims, is characterized by the fact that the light source (1) is pivotable upwards or downwards on the retainer (3) and perpendicularly connected to its horizontal direction. The device, based on claims 1 through 8, is characterized by the fact that the light source is pivotable and placed on a clip-mount. The device, based on one of the previous Claims, is characterized by the fact that the power source (2) is a battery connected or connectable to a light source (1). The device, based on claims 1 through 11, is characterized by the fact that the power source (2) is integrated or integratable in the retainer (3) or the clip-mount (8).
